(19) United States
(12) Patent Application Publication
(10) Pub. No.: US 2005/0080280 A1
(43) Pub. Date: Apr. 14, 2005

(54) Process for producing alkylester of fatty acid in a single-phase continuous process

(76) Inventor: Jeong-Woo Yoo, Uiwaing-si (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/913,300

(22) Filed: Aug. 5, 2004

Related U.S. Application Data
(63) Continuation-in-part of application No. PCT/KR02/00629, filed on Apr. 9, 2002.

(30) Foreign Application Priority Data
Feb. 5, 2002 (KR) 10-2002-0006593

(51) Int. Cl. C07C 51/43

(52) U.S. Cl. 554/174

(57) ABSTRACT

The present invention relates to a process for preparing an alkylester of fatty acid with high purity via one-step continuous process by reacting an animal fat and/or vegetable oil with a lower alcohol in the presence of alkali catalyst by passing through a continuous tubular reactor while maintaining a single-phase, removing residual lower alcohol from the reaction mixture and removing residual glycerin, catalyst, etc. by phase separation. In accordance with the present invention, an alkylester of fatty acid can be produced with a high yield of 97% or more via one-step continuous process in a continuous tubular reactor without any limitation in flow types by reacting an animal fat and/or vegetable oil with a lower alcohol in the presence of alkali catalyst and carrying out a simple separating process.

### RELATED APPLICATIONS

This application claims for the benefit of an earlier filing date under 35 U.S.C. § 365(c) of International Application No. PCT/KR02/00629 filed Apr. 9, 2002, designating the United States and claiming for the benefit of the earlier filing date under 35 U.S.C. § 365(b) of Korean Patent Application No. 2002-0006593 filed Feb. 5, 2002, which is hereby incorporated herein by reference in their entirety. International Application No. PCT/KR02/00629 was published in English as WO 03/066567 A1 on Aug. 14, 2003, and is hereby incorporated herein by reference in its entirety. The present specification supersedes any inconsistencies between the present specification and the references incorporated herein.

### BACKGROUND OF THE INVENTION

1. Field of the Invention

The present invention relates to a process for preparing an alkylester of fatty acid with high purity via one-step continuous process, more specifically, to a process for preparing an alkylester of fatty acid with high purity via one-step continuous process by reacting an animal fat and/or vegetable oil with a lower alcohol in the presence of alkali catalyst by passing through a continuous tubular reactor while maintaining a single-phase, removing residual lower alcohol from the reaction mixture and removing residual glycerin, catalyst, etc. by phase separation.

2. Description of the Related Technology

In general, alkylester of fatty acid is prepared by reacting an animal fat and/or vegetable oil with a lower alcohol in the presence of a homogeneous catalyst of strong base such as sodium hydroxide or strong acid such as sulfuric acid.

As a conventional process using a homogeneous catalyst of strong acid, German Patent No. 1,909,434 discloses transesterification between methylacetate and butylalcohol in the presence of a catalyst of concentrated sulfuric acid at a temperature of 95° C. to 105° C. Harrington has also reported the transesterification reaction, in which vegetable oil from sunflower seed is mixed with 100 or more molar ratio of methanol and reacted in the presence of a catalyst of concentrated sulfuric acid for 3 to 4 hours, to produce methylester of fatty acid with a yield of 40.7% (see: Harrington, Ind. Eng. Chem. Prod. Res. Dev., 1985, 24:314-318).

Meanwhile, transesterification using a homogeneous catalyst of strong base has been also reported in the art(see: B. Freedman, J.A.O.C.S., 1984, 61(10): 1638-1643): for example, European Patent No. 301,634 teaches a process for preparing ester using a hydrophilic strong base catalyst such as KOH, K₂CO₃ and NaOH, inter alia, a process for preparing ester using a catalyst of strong base became commercially available owing to its higher reaction rate than that of using acid catalyst.

In a commercial process employing a catalyst of strong base, animal fat and/or vegetable oil are diluted in a lower alcohol which is several or dozens times as much as oil, then reacted in the presence of a catalyst of sodium hydroxide for 1 to 10 hours to produce a mixture of alkylester of fatty acid and glycerin. And then, a layer of alkylester of fatty acid and a glycerin layer are separated in a separating tower, the glycerin layer is subsequently neutralized with sulfuric acid and the catalyst is removed by way of precipitation and filtration, a filtered solution is transferred to a distillating tower and the lower alcohol is removed by distillation to give glycerin, and the layer of alkylester of fatty acid is washed several times with water, finally to produce alkylester of fatty acid in a drying tower. The said process is not satisfactory in the senses that: the efficiency and productivity are not good since most of the steps using a hydrophilic homogeneous catalyst of base are carried out in separate batch reactors; and, the reactivity is not good because of the high hydrophilicity of catalyst and the low miscibility of catalyst with an animal fat and/or vegetable oil. To solve these problem, needs for continuous process for preparing an alkylester of fatty acid and the development of catalyst improved in terms of reactivity have been continued in the art.

As an example of continuous process for preparing an alkylester of fatty acid, Austrian Patent No. PJ 1105/88(1988) discloses two-step continuous process, in which two continuous stirred tank reactors are linked in a serial manner: methylester of fatty acid is first obtained by mixing an animal fat and/or vegetable oil, methylalcohol and catalyst in the first reactor and a glycerin layer containing catalyst and lower alcohol is removed, and then methylalcohol and catalyst are added and reacted in the second reactor to produce methylester of fatty acid with a yield of 97%. In the said process, reaction is initiated under a condition that oil and methylalcohol form two-phase of liquid/liquid, and the reaction system is changed to a single-phase by the production of diglyceride and monoglyceride, and converted to two-phase again with the increase in the concentrations of hydrophilic glycerin and lipophilic alkylester of fatty acid.

In carrying out the process, vigorous stirring is essential at the beginning and end of the reaction due to the extremely high solubility of catalyst in a hydrophilic material, and a powerful blender should be provided in the reactor to prevent the decrease in the reaction rate or the production yield because most catalyst and significant amount of methanol are dissolved in the glycerin layer. Furthermore, transesterification is reversible even in a case the reactants are mixed well, which leads the two-phase reaction to reach to an equilibrium state in a range of yield of 80 to 90%, therefore, transesterification with two or more steps is essentially required. As a consequence, although the afore-mentioned process is a continuous process firstly introduced for preparing an alkylester of fatty acid, there are drawbacks that the process is complicated and requires two steps, and the reaction rate is low and large facilities are accompanied, because large amount of catalyst and methylalcohol are transferred to the glycerin layer due to the nature of two-phase reaction.

Under the circumstances, efforts for improving the reactivity of catalyst in a continuous process have been continuously made in the art: for example, French Patent No. 1,583,583 discloses a process using Na metal catalyst instead of alkali catalyst, and U.S. Pat. No. 3,853,315 teaches transesterification of vegetable oil by using Na and K.

Particularly, WO 91/05034, EP 409 177 and DE 3925514 by Henkel Inc. a German company, suggest a process for preparing an alkylester of fatty acid with high yield by ranging the catalyst in a layer of lipophilic methylester using a catalyst of sodium methoxide, which is highly soluble in lipophilic material, while preventing the decrease in the efficiency of catalyst and the yield of process. The said process practically realized a yield of about 85% in the first reactor at a temperature of 100° C. or below, and yield of 98% in total through the second reactor after the removal of glycerin and the addition of alcohol and catalyst, by using a multi-step continuous tubular reactor with two or more serially linked continuous tubular reactors and facilities for separating glycerin and supplying lower alcohol and catalyst between the reactors, and by employing alcohol/oil in a molar ratio of 4.5 to 7.5. The said patents have contributed to a yield increase at the latter part of reaction through controlling the migration of catalyst into the glycerin layer by using a catalyst of sodium methoxide. However, a flow rate in the continuous tubular reactor should be required to maintain Reynold's number of above 2300 to minimize the decrease in catalytic efficiency while increasing the mixing power in the continuous tubular reactor due to the two-phase nature of the reaction system. And, two-step reactor for transesterification should be further provided to prepare the alkylester of fatty acid with high yield.

Recently, the usage of high-purity alkylester of fatty acid, inter alia, methylester of fatty acid as bio-diesel has been rapidly increased. To pass the revelent European standard, the purity of methylester of fatty acid for bio-diesel should be more than 96.5%, which naturally motivated the studies on a process for preparing a methylester of fatty acid with a high yield of 96.5% or more. For example, Japanese patent laid-open publication No. 10-182518 discloses a process for preparing methylester of fatty acid with a yield of 96.5% via one-step process from decayed edible oil, in which the molar ratio of alcohol/oil is controlled in a range of 4.3 to 6.6, and reaction is carried out for 15 min by using a catalyst of sodium hydroxide. However, the said process has revealed shortcomings that: the yield is highly dependent on the flow rate, since the process is performed via two-phase reaction; and, high-purity alkylester of fatty acid cannot be produced in a continuous tubular reactor without special techniques.

Under the circumstances, there are strong reasons for exploring and developing a process for preparing an alkylester of fatty acid with high purity by employing a continuous tubular reactor via one-step continuous process.

### SUMMARY OF THE INVENTION

One aspect of the present invention provides a method of conducting a transesterification reaction between alkyl alcohol and a glyceride. The method comprises: continuously supplying a substance containing a glyceride having at least one fatty acid moiety; continuously adding alkyl alcohol and a metal hydroxide catalyst to the substance, wherein the substance, alkyl alcohol and metal hydroxide are dissolved in each other, thereby creating a solution, in which the transesterification reaction may be initiated; continuously flowing the solution through a tubular reactor while preventing the solution from undergoing phase separation as the transesterification reaction continues to form an alkylester of the fatty acid and a glycerine; separating the alkylester of the fatty acid from the resulting solution.

In the above-described method, the transesterification reaction may be conducted while the solution may be flowing at a Reynold's number below 2100. The transesterification reaction may be conducted while solution may be flowing at a Reynold's number above 2100. The method may be conducted in an industrial scale. The continuous adding may be conducted at a temperature about 40° C. or higher. The phase separation may be prevented by setting a pressure of the solution in the tubular reactor sufficient to prevent evaporation of the alkyl alcohol and glycerin at a given temperature. The temperature of the solution in the tubular reactor may be selected from about 60° C. to about 150° C. The pressure of the solution in the tubular reactor may be selected from about 1 atm to about 10 atm.

Still in the above-described method, the substance containing the glyceride may be at least one of animal fat and vegetable oil. The alkyl alcohol may be selected from the group consisting of methyl alcohol, ethyl alcohol, propyl alcohol, n-butyl alcohol, 2-ethyl alcohol, and a mixture of two or more of the foregoing. After addition of the alkyl alcohol, the alkyl alcohol is present in the solution from about 6 to about 60 (mole/mole) times that of the glyceride. After addition of the metal hydroxide catalyst, the catalyst is present in the solution in an amount from about 0.1 to about 2% (w/w) of the amount of the glyceride. The separating of the alkylester of the fatty acid may further comprise removing residual alkyl alcohol from the resulting solution. The separating of the alkylester of the fatty acid may further comprise: allowing phase separation in the resulting solution, thereby forming a lipophylic layer and a hydrophilic layer; collecting the lipophilic layer containing the alkylester of the fatty acid; and separating the alkylester of the fatty acid from the lipophilic layer.

Another aspect of the present invention provides a method of producing an alkylester of a fatty acid. The method comprises: mixing an alkyl alcohol, a catalyst and a glyceride at a temperature sufficient to dissolve the alkyl alcohol, catalyst and glyceride in each other, thereby creating a single-phase mixture and initiating a transesterification reaction between the glyceride and alkyl alcohol in the single-phase mixture, wherein the glyceride has at least one fatty acid moiety; maintaining the mixture as a single-phase throughout the reaction by selecting a temperature and a pressure sufficient to prevent phase separation in the mixture as the transesterification reaction may be carried out; and separating an alkylester of the fatty acid from the mixture.

In the above-described method, substantial part of the transesterification reaction is carried out while the single-phase mixture is being transferred through a continuous tubular reactor. The method may be conducted in a continuous mode, in which the mixture is substantially constantly flowing through a continuous reactor. The transesterification reaction may be conducted while mixture is flowing at a Reynold's number below 2100. The transesterification reaction may be conducted while mixture is flowing at a Reynold's number above 2100. The method may be conducted in a batch mode. The method may be conducted in an industrial scale. The mixing may be conducted at a temperature about 40° C. or higher. The phase separation may be prevented by selecting a pressure sufficient to prevent evaporation of the alkyl alcohol and glycerin at a given temperature.

Still in the above-described method, the temperature may be selected from about 60° C. to about 150° C. The temperature may be selected from about 70° C. to about 150° C. The pressure may be selected from about 1 atm to about 10 atm. The glyceride may be in the form of animal fat or vegetable oil. The alkyl alcohol may be selected from the group consisting of methyl alcohol, ethyl alcohol, propyl alcohol, n-butyl alcohol, 2-ethyl alcohol, and a mixture of two or more of the foregoing. After the mixing, the alkyl alcohol may be present in the single-phase mixture from about 6 to about 60 (mole/mole) times that of the glyceride. The catalyst may be present in the single-phase mixture in an amount from about 0.1 to about 2% (w/w) of the amount of the glyceride. The catalyst may be a metal hydroxide. The separating of an alkylester of the fatty acid may further compriseremoving residual alkyl alcohol from the reaction mixture. The separating of an alkylester of the fatty acid further comprises: allowing phase separation in the reaction mixture, thereby forming a lipophylic layer and a hydrophilic layer; collecting the lipophilic layer containing the alkylester of the fatty acid; and separating the alkylester of the fatty acid from the lipophilic layer.

Still another aspect of the present invention provides an alkylester of a fatty acid produced by the above-described methods.

### BRIEF DESCRIPTION OF THE DRAWINGS

FIG. 1 depicts a diagram showing a process for preparing an alkylester of fatty acid via one-step continuous process of the present invention

### EXPLANATION OF SYMBOLS IN THE MAJOR PARTS OF FIGURE

\mathring{1}, \mathring{2} . . . heat exchanger
\mathring{3}, \mathring{4} . . . booster pump
\mathring{5} . . . blender
\mathring{6} . . . continuous tubular reactor
\mathring{7} . . . evaporator
\mathring{8}, \mathring{9} . . . separating apparatus

### DETAILED DESCRIPTION OF THE INVENTION

The present inventors have made an effort to develop a process for preparing an alkylester of fatty acid with high purity via one-step continuous process, and found that an alkylester of fatty acid with a high purity of 98% or more can be prepared via one-step continuous process by reacting an animal fat and/or vegetable oil with a lower alcohol in the presence of alkali catalyst by passing through a continuous tubular reactor while maintaining a single-phase.

A process for preparing an alkylester of fatty acid with high purity of the present invention comprises the steps of:
(i) mixing an animal fat and/or vegetable oil with a lower alcohol in the presence of alkali catalyst to give a singe-phase, reacting the mixture in one-step continuous tubular reactor while maintaining the singe-phase, to obtain a reaction mixture of alkylester of fatty acid, glycerin, lower alcohol and catalyst;
(ii) removing residual lower alcohol from the reaction mixture obtained in (i); and,
(iii) separating the mixture obtained in (ii) into a layer of alkylester of fatty acid and a glycerin layer containing glycerin and catalyst, removing residual glycerin layer to prepare an alkylester of fatty acid.

The process for preparing an alkyester of fatty acid with high purity, if necessary, may further comprise a step of removing insoluble solid materials from the alkylester of fatty acid obtained in Step(iii).

The process for preparing an alkylester of fatty acid is further illustrated in more detail, in accordance with the steps as followings.

Step 1: Transesterification Via One-Step Continuous Tubular Reaction

An animal fat and/or vegetable oil is mixed with alkali catalyst dissolved in a lower alcohol to give a single-phase, and the mixture is reacted in one-step continuous tubular reactor while maintaining the single-phase to obtain a reaction mixture of alkylester of fatty acid, glycerin, lower alcohol and catalyst:

The animal fat and/or vegetable oil includes soybean oil, rape oil, sunflower seed oil, castor oil, corn oil, palm oil, beef tallow and mixture thereofs, where C₈˜C₃₀ saturated or unsaturated fatty acids such as stearic acid, oleic acid, linoleic acid, linolenic acid, palmitic acid, myristic acid, arachidic acid and lauric acid are present in a form of mono-, di- or triglyceride, linked to glycerin.

The lower alcohol includes methylalcohol, ethylalcohol, propylalcohol, n-butylalcohol, 2-ethylalcohol and mixture thereofs. The amount of lower alcohol, as one of parameters to give a single-phase, is controlled preferably in a range of 6 to 60 times(in molar ratio) as much as the animal fat and/or vegetable oil. Less than 6 times and more than 60 times of alcohol are less preferable, since the former lowers a conversion ratio of oil to ester and the latter requires more energy to separate lower alcohol after reaction.

The alkali catalyst is used in a range of 0.1 to 10% (w/w) of the animal fat and/or vegetable oil, preferably 0.1 to 3% (w/w),more preferably 0.3 to 2% (w/w). The catalyst includes, for example, metal hydroxide such as potassium hydroxide(KOH), sodium hydroxide(NaOH), lithium hydroxide(LiOH), rubidium hydroxide(RbOH) or cesium hydroxide(CsOH); metal alkoxide such as sodium methoxide(CH₃ONa), sodium ethoxide(CH₃CH₂ONa), potassium methoxide(CH₃OK), potassium ethoxide(CH₃CH₂OK), lithium methoxide(CH₃OLi), lithium ethoxide(CH₃CH₂OLi); multivalent metal alkoxide such as dibutoxide-dibutyl tin(C₁₆H₃₆O₂Sn), tin butoxide(C₁₆H₃₆O₄Sn), titanium butoxide(C₁₆H₃₆O₄Ti), zirconium butoxide(C₁₆H₃₆O₄Zr), titanium propoxide(C₁₂H₂₈O₄Ti), zirconium propoxide(C₁₂H₂₈O₄Zr), titanium ethoxide(C₈H₂₀O₄Ti), zirconium ethoxide(C₈H₂₀O₄Zr), titanium methoxide(C₄H₁₂O₄Ti); and, ammonium hydroxide of tetrabutylammonium hydroxide([CH₃(CH₂)₂CH₂]₄NOH).

In carrying out the present invention, mixing and reacting of the reactants is made at a temperature range of 60 to 150° C., depending on the amount of used alcohol. The temperature is preferably maintained above 70° C. in a soybean oil/methylalcohol reaction system. Though high reaction temperature is required to maintain a high reaction rate in a completely mixed state, it is preferred to keep the temperature not higher than 150° C. to avoid potential carbonization and saponification of fat and/or oil.

In carrying out the present invention, a pressure is maintained in a range of 1 to 10 atm for preventing alcohol from evaporation and maintaining a single-phase of reactants. The higher the pressure is, the easier the single-phase is made. However, it is preferable that the pressure is maintained to the minimum required for preventing the evaporation of alcohol and maintaining the single-phase at a certain temperature since much higher pressure increases the working expenses.

In carrying out the present invention, the major parameters for subjecting reactants and a reaction mixture to a state of single-phase includes a ratio of lower alcohol to animal fat and/or vegetable oil, temperature and pressure. After selecting a reaction temperature, the amount of lower alcohol is determined, which is necessary for initiation of reaction and preventing the phase separation of reaction products, i.e., alkylester of fatty acid and glycerin. Practically, the amount of residual lower alcohol necessary for preventing the phase separation is determined based on three-phase solubility curve of three materials, i.e., alkylester of fatty acid, glycerin and alcohol, and from which the total amount of alcohol to alkylester of fatty acid is determined. The amount of alcohol used at a certain temperature is changed depending on the kind of alcohol, and ranges in a molar ratio of 6 or more to animal fat and/or vegetable oil, preferably in a range of 10 or more. For example, in case of methanol/soybean oil reaction system, the molar ratio of methanol to soybean oil is 25.5 or more at 60° C., and 14.7 or more at 80° C., respectively. The pressure is maintained in a range of 1 to 10 atm to prevent lower alcohol from evaporation at a certain temperature and a certain amount of lower alcohol.

Transesterification of the present invention is made in a single-phase unlike prior art. The reaction of an animal fat and/or vegetable oil and a lower alcohol is carried out via a novel reaction mechanism: 1) alkali catalyst is linked to ester group of fat and/or oil, which is relatively more acidic than the lower alcohol, to give an intermediate with increased reactivity; and, 2) transesterification between alcohol and reactive ester group of oil is followed(see: Reaction Scheme 1).

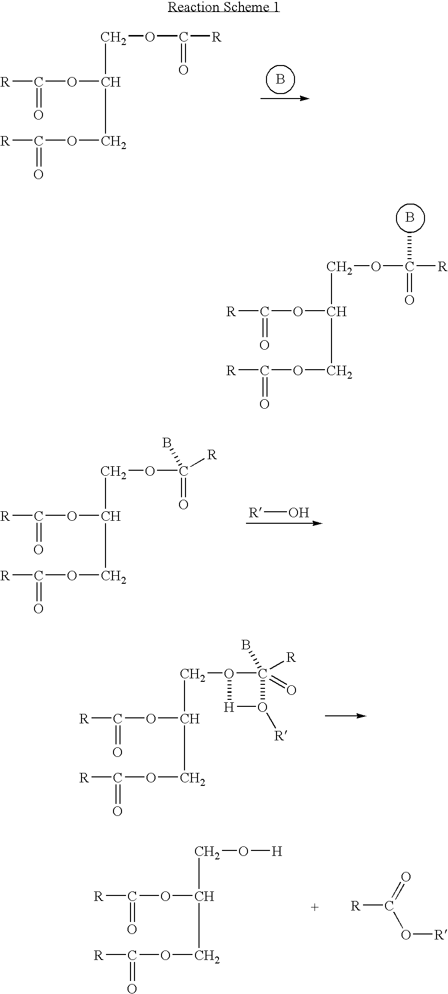

In accordance with the conventional process, where phase separation takes place at the beginning and end of reaction, the reaction is carried out via formation of alkoxide between lower alcohol and alkali catalyst and transesterification between alkoxide and ester. In the prior art, since the alkali catalyst is dissolved only in a hydrophilic component, the reaction is made only in an interface between the two phases. Accordingly, at the beginning of reaction, the catalyst exists only in a layer of lower alcohol, and the reaction rate abruptly drops without vigorous stirring, and long reaction time is required in a tubular reactor with low mixing efficiency and catalyst and lower alcohol are migrated to a layer of glycerin produced at the end of the reaction, which, in turn, results in a decrease in the concentrations of catalyst and lower alcohol needed to be reacted. As a consequence, high-yield of alkylester of fatty acid cannot be realized in the prior art.

The present invention successfully solved the said problems caused by two-phase reaction through the transesterification employing a single-phase reaction mechanism shown in Reaction Scheme 1.

In accordance with the present invention, besides the aspects of catalytic efficiency, an improvement in terms of yield can be accomplished by minimizing the reverse reaction by three alcohol groups of glycerin, by way of blocking the phase separation of alkylester of fatty acid and glycerin. That is, in a case that hydrophilic glycerin with three polar alcohol groups is forced to be mixed with lipophilic phase, the glycerin, due to rare polar groups in the vicinity of the molecule, forms pseudo-ring depicted in chemical formula (I), which lowers its polarity, and decreases reverse reaction.

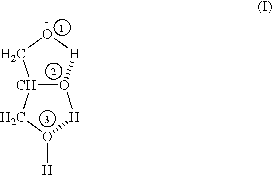

That is, only oxygen \mathring{1} of glycerin, in the lipophilic environment, has a reactivity, which decreases the number of alcohol groups in the glycerin molecule capable of driving reverse reaction. Further, the reactivity can be decreased compared with primary alcohol or alcohol groups of glycerin in hydrophilic phase, because the hydrogen of alcohol group \mathring{1} of glycerin can be linked to adjacent oxygen in the same molecule by hydrogen bond. Accordingly, the reverse reaction of glycerin and alkylester of fatty acid can be minimized, which, in turn, makes it possible to produce alkylester of fatty acid with a high yield of 97% or more even in one-step process.

Transesterification in the present invention preferably proceeds in a continuous tubular reactor, without accompanying phase separation, which provides an excellent mixing nature even in a continuous tubular reactor with poor mixing efficiency. Accordingly, in comparison with German Patent No. 3925514, which requires to maintain a turbulent flow of above Reynold's number 2300 to maximize the mixing of reactants in a continuous tubular reactor, the present invention has an advantage of realizing a homogeneous reaction in a laminar flow domain and in a turbulent flow domain as well.

Step 2: Removal of Lower Alcohol

Lower alcohol is removed from the reaction mixture obtained in Step 1: the method of removing the lower alcohol, not limited thereto, includes distillation(simple distillation, distillation under reduced pressure, fractional distillation, distillation using thin layer distiller) etc., which are conventional in the art.

According to the conventional methods, the reaction mixture can be separated into a mixed layer of alkylester of fatty acid and lower alcohol and a mixed layer of glycerin, lower alcohol and catalyst, in which two separate apparatuses for the removal of the residual lower alcohol in each of the layers are essentially required. Further, there may exist a problem that glycerin, catalyst and soap components are dissolved into the mixed layer of alkylester of fatty acid and lower alcohol. In the present invention, the removal of lower alcohol from the single-phase mixture obtained after transesterification is first carried out, which provides the following advantages: the process is performed in a simple manner; and, the dissolution of glycerin, catalyst and soap components into a layer of alkylester of fatty acid, which may be caused by the co-existence of alkylester of fatty acid and lower alcohol, can be prevented.

Step 3: Phase Separation of Mixture and Preparation of Alkylester of Fatty Acid

Alkylester of fatty acid is prepared by separating the mixture obtained in Step 2 into a layer of alkylester of fatty acid and a glycerin layer containing glycerin, catalyst and soap components in a form of precipitate, and removing the glycerin layer therefrom: the method of separating the layer, not limited thereto, includes simple separation, liquid/liquid centrifuge, etc., which are conventional in the art.

In the present invention, catalyst is present only in a glycerin layer because residual lower alcohol is removed prior to the separation of an ester layer and a glycerin layer. Accordingly, catalyst can be removed together with the removal of glycerin layer, and small amounts of soap components produced during transesterification, can be removed through simple separation step because they are not dissolved into the layer of alkylester of fatty acid. As a consequence, alkylester of fatty acid with high purity can be prepared.

A process for preparing alkylester of fatty acid in the present invention may further comprise a step of removing insoluble solid materials from the alkylester of fatty acid obtained in Step 3, in a case that the insoluble solid materials such as soap, etc. exist in the layer of alkylester of fatty acid.

The present invention is further illustrated in the following examples, which should not be taken to limit the scope of the invention.

### REFERENCE EXAMPLES 1 TO 7

### Determination of Mixing Ratio of Animal Fat and/or Vegetable Oil to Lower Alcohol

In carrying out the present invention, a reaction system is maintained in a single-phase to produce an alkylester of fatty acid with high purity, for this purpose, it is critical to subject the reaction products, i.e., lipophilic alkylester of fatty acid and hydrophilic glycerin, to a state of single-phase to the end of reaction. Therefore, the point that the final reaction products reach to a single-phase was determined, while varying the concentrations of lower alcohol at a certain temperature, and the amount of lower alcohol to animal fat and/or vegetable oil at the initial point was determined therefrom, for the purpose of maintaining a single-phase of reaction products to the end of reaction.

First, 36 g of methylester of fatty acid(98.5%) and 4 g of glycerin(99.5%) produced from soybean oil were injected into a 250 ml reactor with fixed-quantity injection device, temperature and pressure controller, and stirrer, then, the temperature of the reactor was elevated to a certain point shown in Table 1 below. Methanol was added gradually under a condition of maintaining the temperature, and the concentration of methanol was determined when the mixture was turned into a single-phase, and the minimum amount of methanol mixed with soybean oil was determined at a certain temperature of reaction in order to adjust the concentration ratio of methanol/methylester of fatty acid after transesterification to the said ratio of concentration, whose results are shown in Table 1 below.

TABLE 1

| | | | Molar ratio of |
|---|---|---|---|
| | | Concentration of methanol | methanol/soybean oil to |
| Reference | Temp | in a reactor conferring a | maintain a single-phase |
| Example | (° C.) | single-phase | of reaction |
| 1 | 40 | 57.8% | 45.5 |
| 2 | 50 | 47.1% | 35.0 |
| 3 | 60 | 39.9% | 25.5 |
| 4 | 70 | 32.9% | 19.2 |
| 5 | 75 | 28.4% | 16.4 |
| 6 | 80 | 24.9% | 14.7 |
| 7 | 85 | 21.7% | 13.3 |

As can be seen in Table 1, it was determined that molar ratio of methanol/soybean oil necessary to maintain a single-phase of reaction varies depending on the temperature, and 10 or more molar ratio of methanol to soybean oil was required thereto.

In addition, 36 g of ethyl ester of fatty acid(98.5%) and 4 g of glycerin(99.5%) produced from soybean oil were injected into a 250 ml reactor with fixed-quantity injection device, temperature and pressure controller, and stirrer, then, the temperature of the reactor was elevated to a certain point shown in Table 1-2 below. Ethanol was added gradually under a condition of maintaining the temperature, and the concentration of ethanol was determined when the mixture was turned into a single-phase, and the minimum amount of ethanol mixed with soybean oil was determined at a certain temperature of reaction in order to adjust the concentration ratio of ethanol/ethylester of fatty acid after transesterification to the said ratio of concentration, whose results are shown in Table 2 below.

TABLE 1-2

| | | Concentration of ethanol | Molar ratio of ethanol/ |
|---|---|---|---|
| | | in a reactor | soybean oil to |
| Reference | Temp | conferring a single- | maintain a single-phase of |
| Example | (° C.) | phase | reaction |
| 8 | 40 | 51.5% | 28.2 |
| 9 | 60 | 35.1% | 19.2 |
| 10 | 80 | 28.9% | 15.8 |

As can be seen in Table 1-2, it was determined that molar ratio of ethanol/soybean oil necessary to maintain a single-phase of reaction varies depending on the temperature, and 10 or more molar ration of ethanol to soybean oil was required thereto

### EXAMPLE 1

### Preparation of Alkylester of Fatty Acid with High Purity in a Continuous Tubular Reactor

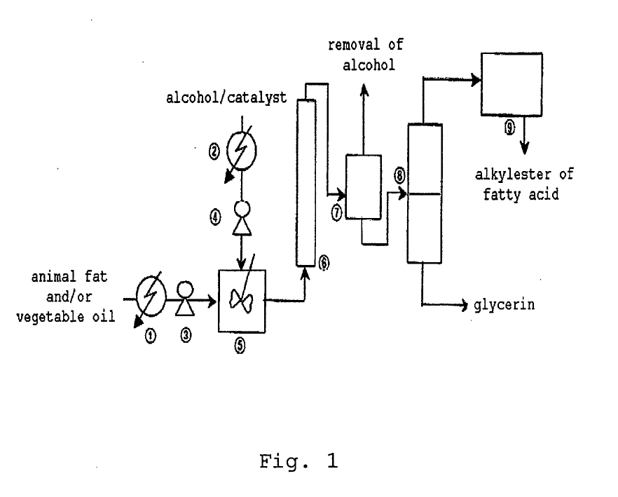

As depicted in FIG. 1, animal fat and/or vegetable oil heated at about 100° C. in a heat exchanger 1, and methylalcohol, in which sodium hydroxide is dissolved in a ratio of 0.5% (w/w) to the animal fat and/or vegetable oil, heated at about 60° C. in a heat exchanger 2 were injected into 15 L of a blender equipped with stirring bar at a uniform speed of 81 kg/hr (an industrial scale which is substantially larger than a laboratory scale) using a booster pump, while maintaining the temperature and pressure of the blender at 78° C. and 5 atm, respectively. Reactants were left to stand in the blender for 30 sec to reach to a single-phase, which was then transferred to a continuous tubular reactor 6. A tubular reactor of duct-form was provided in a thermostat facility maintaining a temperature of 80° C., whereby preventing a decrease in the temperature of reactants, and the retention time of the mixture in the reactor was adjusted to 15 min in total by passing the mixture through a reactor with 4 cm of diameter, 35.8 m of total length at a speed of 180 L/hr. After completing the reaction, the final mixture from the reactor was immediately directed to an evaporator 7 to remove methylalcohol, then transferred to a separator 8 in which a glycerin layer containing catalyst and a layer of methylester of fatty acid were separated, respectively. In a case that insoluble solid materials are present in the layer of methylester of fatty acid, they were further removed in a separator 9. The methylester of fatty acid thus prepared was analyzed by the aid of Gas Chromatography(HP6890, FID) equipped with BPX5 column. The result showed that the conversion ratio of alkylester of fatty acid was 98.5%. In this example, the physicochemical parameters of a mixture in the reactor were as follows(see: Table 2).

TABLE 2

| Physicochemical parameters of a mixture in | |
|---|---|
| a continuous tubular reactor | Numerical value |
| $Density , p ( \frac{1}{p} = {\sum }_{i} \frac{{w}_{i}}{{p}_{i}} ) 80 ° C . , 5 atm$ | 850 kg/m³ |
| $Viscosity , n ( lnn = {\sum }_{i} {x}_{i} {n}_{i} ) 80 ° C . , 5 atm$ | 0.665 cp |
| Volume flow rate | 0.18 m³/hr |
| $Reynold ' s number ( {Re}_{D} = \frac{4 Qp}{πDn} )$ | 712 |
| Yield of methylester of fatty acid | 98.5% |

As can be seen in Table 2, the yield of methylester of fatty acid was 98.5% even in a laminar flow domain that the Reynold's number(Re_{D}) is below 2100.

### COMPARATIVE EXAMPLES 1 AND 2

### Reaction in Case of Two-Phase of Reactants

Methylester of fatty acid was prepared similarly as in Example 1 except that a molar ratio(or weight ratio) of methanol to animal fat and/or vegetable oil was different from each other: first, soybean oil and catalyst-methanol solution were injected into a blender at a speed of 130 kg/hr and 30 kg/hr, respectively, at the same temperature as in Example 1, and reacted in a continuous tubular reactor with 4 cm of diameter. In carrying out Comparative Example 1, the length of reactor in total was 35.8 m, to maintain 15 min of retention time in the reactor. In carrying out Comparative Example 2, the length of reactor in total was 71.6 m, allowing 30 min of retention time in the reactor. The results of Comparative Examples 1 and 2 revealed that: the conversion ratios of methylester of fatty acid were 64% and 77%, respectively; and, two-phase reaction employing a continuous tubular reactor cannot provide methylester of fatty acid with a high purity of 97% or more via one-step continuous process.

### EXAMPLE 2

### Preparation of Alkylester of Fatty Acid with High Purity in a Single Continuous Turbulent Tubular Reactor

Methylester of fatty acid was prepared similarly as in Example 1 except that Reynold's number in a continuous tubular reactor was changed by adjusting the inner diameter of the continuous tubular reactor to 1.25 cm and the total length to 349 m. The results revealed that the conversion ratio of methylester was 98.6%.

In carrying out this Example, the physicochemical parameters of a mixture in the reactor were as follows(see: Table 3).

TABLE 3

| Physicochemical parameters of a mixture in a | |
|---|---|
| continuous tubular reactor | Numerical value |
| $Density , p ( \frac{1}{p} = {\sum }_{i} \frac{{w}_{i}}{{p}_{i}} ) 80 ° C . , 5 atm$ | 850 kg/m³ |
| $Viscosity , n ( lnn = {\sum }_{i} {x}_{i} {n}_{i} ) 80 ° C . , 5 atm$ | 0.665 cp |
| Volume flow rate | 0.18 m³/hr |
| $Reynold ' s number ( {Re}_{D} = \frac{4 Qp}{πDn} )$ | 2279 |
| Yield of methylester of fatty acid | 98.6% |

As can be seen in Table 3, the yield of methylester of fatty acid was 98.6% even in a turbulent flow domain that the Reynold's number(Re_{D}) is above 2100.

### EXAMPLES 3 TO 7

Methylester of fatty acid was prepared analogously as in Example 1 except for employing different alkali catalysts. The yield of methylester of fatty acid and the catalysts are shown in Table 4.

TABLE 4

| | | Yield of methylester of |
|---|---|---|
| Example | Catalyst | fatty acid |
| 3 | sodium hydroxide(NaOH) | 97.3% |
| 4 | sodium methoxide(CH₃ONa) | 98.2% |
| 5 | zirconium butoxide(C₁₆H₃₆O₄Zr) | 97.2% |
| 6 | dibutoxide-dibutyl tin(C₁₆H₃₆O₂Sn) | 97.3% |
| 7 | tetrabutylammonium | 97.6% |
| | hydroxide([CH₃(CH₂)₂CH₂]₄NOH) | |

As can be seen in the above Table 4, it was clearly demonstrated that alkylester of fatty acid with a high yield of 97% or more can be prepared by reacting alcohol with animal fat and/or vegetable oil in the presence of alkali catalyst such as metal hydroxide, metal methoxide, multivalent metal alkoxide, ammonium hydroxide, etc.

### EXAMPLES 8 TO 12

Methylester of fatty acid was prepared in a similar fashion as in Example 1 except for employing different lower alcohols. The yield of methylester of fatty acid depending on the kind and amount of lower alcohol are shown in Table 5.

TABLE 5

| | | Molar ratio of alcohol | |
|---|---|---|---|
| | | to animal fat and/or | Yield of alkylester |
| Example | Lower alcohol | vegetable oil | of fatty acid |
| 8 | methylalcohol | 27.8 | 98.5 |
| 9 | ethylalcohol | 19.8 | 98.0 |
| 10 | propylalcohol | 16.5 | 97.8 |
| 11 | butylalcohol | 14.8 | 97.1 |
| 12 | 2-ethylhexanol | 14.2 | 97.2 |

As can be seen in the above Table 5, alkylester of fatty acid can be obtained with a high yield of 97% or more by maintaining the reaction in a single-phase by way of controlling the ratio of alcohol to animal fat and/or vegetable oil depending on the kind of alcohol.

As clearly illustrated and demonstrated as above, the present invention provides a process for preparing an alkylester of fatty acid with high purity via one-step continuous process by reacting an animal fat and/or vegetable oil with a lower alcohol in the presence of alkali catalyst by passing through a continuous tubular reactor while maintaining a single-phase. In accordiance with the present invention, all of the used catalysts can be efficiently participated in esterification and reversible reaction can be prevented by decreasing the reactivity of alcohol groups of glycerin, which allows a high yield production of 97% or more of alkylester of fatty acid via one-step process even in a continuous tubular reactor with poor mixing efficiency. Further, residual lower alcohol can be removed prior to the separation of a glycerin layer, which makes all of the used catalysts reside in the glycerin layer, and soap components produced in a small amount as a by-product in the course of preparation, can be precipitated in a layer of alkylester of fatty acid, which can afford the simplified separation with high efficiency and the decrease in the working expenses.

While the present invention has been shown and described with reference to the particular embodiments, it will be apparent to those skilled in the art that many changes and modifications may be made without departing from the spirit and scope of the invention as defined in the claims. Accordingly, the substantial scope of the present invention is defined as the attached claims and their equivalents.

1. A method of conducting a transesterification reaction between alkyl alcohol and a glyceride, the method comprising:
continuously supplying a substance containing a glyceride having at least one fatty acid moiety;
continuously adding alkyl alcohol and a metal hydroxide catalyst to the substance, wherein the substance, alkyl alcohol and metal hydroxide are dissolved in each other, thereby creating a solution, in which the transesterification reaction is initiated;
continuously flowing the solution through a tubular reactor while preventing the solution from undergoing phase separation as the transesterification reaction continues to form an alkylester of the fatty acid and a glycerine;
separating the alkylester of the fatty acid from the resulting solution.

2. The method of claim 1, wherein the transesterification reaction is conducted while the solution is flowing at a Reynold's number below 2100.

3. The method of claim 1, wherein the transesterification reaction is conducted while solution is flowing at a Reynold's number above 2100.

4. The method of claim 1, wherein the method is conducted in an industrial scale.

5. The method of claim 1, wherein continuous adding is conducted at a temperature about 40° C. or higher.

6. The method of claim 1, wherein phase separation is prevented by setting a pressure of the solution in the tubular reactor sufficient to prevent evaporation of the alkyl alcohol and glycerin at a given temperature.

7. The method of claim 1, wherein the temperature of the solution in the tubular reactor is selected from about 60° C. to about 150° C.

8. The method of claim 1, wherein the pressure of the solution in the tubular reactor is selected from about 1 atm to about 10 atm.

9. The method of claim 1, wherein the substance containing the glyceride is at least one of animal fat and vegetable oil.

10. The method of claim 1, wherein the alkyl alcohol is selected from the group consisting of methyl alcohol, ethyl alcohol, propyl alcohol, n-butyl alcohol, 2-ethyl alcohol, and a mixture of two or more of the foregoing.

11. The method of claim 1, wherein after addition of the alkyl alcohol, the alkyl alcohol is present in the solution from about 6 to about 60 (mole/mole) times that of the glyceride.

12. The method of claim 1, wherein after addition of the metal hydroxide catalyst, the catalyst is present in the solution in an amount from about 0.1 to about 2% (w/w) of the amount of the glyceride.

13. The method of claim 1, wherein separating of the alkylester of the fatty acid further comprises removing residual alkyl alcohol from the resulting solution.

14. The method of claim 1, wherein separating of the alkylester of the fatty acid further comprises:
allowing phase separation in the resulting solution, thereby forming a lipophylic layer and a hydrophilic layer;
collecting the lipophilic layer containing the alkylester of the fatty acid; and
separating the alkylester of the fatty acid from the lipophilic layer.

15. An alkylester of a fatty acid produced by the method of claim 1.

16. A method of producing an alkylester of a fatty acid, comprising:
mixing an alkyl alcohol, a catalyst and a glyceride at a temperature sufficient to dissolve the alkyl alcohol, catalyst and glyceride in each other, thereby creating a single-phase mixture and initiating a transesterification reaction between the glyceride and alkyl alcohol in the single-phase mixture, wherein the glyceride has at least one fatty acid moiety;
maintaining the mixture as a single-phase throughout the reaction by selecting a temperature and a pressure sufficient to prevent phase separation in the mixture as the transesterification reaction is carried out; and
separating an alkylester of the fatty acid from the mixture.

17. The method of claim 16, wherein substantial part of the transesterification reaction is carried out while the single-phase mixture is being transferred through a continuous tubular reactor.

18. The method of claim 16, wherein the method is conducted in a continuous mode, in which the mixture is substantially constantly flowing through a continuous reactor.

19. The method of claim 18, wherein the transesterification reaction is conducted while mixture is flowing at a Reynold's number below 2100.

20. The method of claim 18, wherein the transesterification reaction is conducted while mixture is flowing at a Reynold's number above 2100.

21. The method of claim 16, wherein the method is conducted in a batch mode.

22. The method of claim 16, wherein the method is conducted in an industrial scale.

23. The method of claim 16, wherein mixing is conducted at a temperature about 40° C. or higher.

24. The method of claim 16, wherein phase separation is prevented by selecting a pressure sufficient to prevent evaporation of the alkyl alcohol and glycerin at a given temperature.

25. The method of claim 16, wherein the temperature is selected from about 60° C. to about 150° C.

26. The method of claim 16, wherein the temperature is selected from about 70° C. to about 150° C.

27. The method of claim 16, wherein the pressure is selected from about 1 atm to about 10 atm.

28. The method of claim 16, wherein the glyceride is in the form of animal fat or vegetable oil.

29. The method of claim 16, wherein the alkyl alcohol is selected from the group consisting of methyl alcohol, ethyl alcohol, propyl alcohol, n-butyl alcohol, 2-ethyl alcohol, and a mixture of two or more of the foregoing.

30. The method of claim 16, wherein after the mixing, the alkyl alcohol is present in the single-phase mixture from about 6 to about 60 (mole/mole) times that of the glyceride.

31. The method of claim 16, wherein the catalyst is present in the single-phase mixture in an amount from about 0.1 to about 2% (w/w) of the amount of the glyceride.

32. The method of claim 16, wherein the catalyst is a metal hydroxide.

33. The method of claim 16, wherein separating of an alkylester of the fatty acid further comprises removing residual alkyl alcohol from the reaction mixture.

34. The method of claim 16, wherein separating of an alkylester of the fatty acid further comprises:
allowing phase separation in the reaction mixture, thereby forming a lipophylic layer and a hydrophilic layer;
collecting the lipophilic layer containing the alkylester of the fatty acid; and
separating the alkylester of the fatty acid from the lipophilic layer.

35. An alkylester of a fatty acid produced by the method of claim 16.
